(19) United States
(12) Patent Application Publication
(10) Pub. No.: US 2013/0095322 A1
(43) Pub. Date: Apr. 18, 2013

(54) NOVEL YELLOW PIGMENT COMPOSITION, AND METHOD FOR PRODUCING YELLOW PIGMENT MICROPARTICLES

(71) Applicants: Masaki Maekawa, Izumi-shi (JP); Daisuke Honda, Izumi-shi (JP); Masakazu Enomura, Izumi-shi (JP)

(72) Inventors: Masaki Maekawa, Izumi-shi (JP); Daisuke Honda, Izumi-shi (JP); Masakazu Enomura, Izumi-shi (JP)

(73) Assignee: M. TECHNIQUE CO., LTD., Izumi-shi, Osaka (JP)

(21) Appl. No.: 13/704,129

(22) PCT Filed: May 20, 2011
(86) PCT No.: PCT/JP2011/061694

§ 371 Date: Dec. 13, 2012

(30) Foreign Application Priority Data
Jun. 16, 2010 (JP) 2010-137777

(51) Int. Cl. C07D 403/04; C07D 235/06; C07C 245/06

(52) U.S. Cl. CPC C07D 403/04; C07C 245/06; C07D 235/06; Y10S 977/773; Y10S 977/788; Y10S 977/834; B82Y 40/00
USPC 428/402; 544/300; 534/885; 548/304.4; 264/8; 977/773; 977/788; 977/834

(57) ABSTRACT

Disclosed are: a yellow pigment composition which contains at least one kind of yellow pigment microparticle having excellent transmission characteristics; and a method for producing the yellow pigment microparticle. Specifically disclosed are: a yellow pigment composition which contains at least one kind of yellow pigment microparticle that are characterized in that the difference between the maximum transmittance (Tmax) and the minimum transmittance (Tmin), namely (Tmax−Tmin) is 80% or more in the transmission spectrum at 350-800 nm; and a method for producing the yellow pigment microparticle.

### TECHNICAL FIELD

The present invention relates to a novel yellow pigment composition, and a method for producing yellow pigment microparticles.

### BACKGROUND ART

Materials to be used for coloring, coloring materials, are classified roughly into groups of dye and pigment. Generally speaking, dye is superior to pigment in color characteristics such as chromic and transparent properties and coloring power. However, dye is inferior to pigment in water resistance and heat resistance, and durability such as light resistance and weatherability; and thus, it is often difficult to secure stability which lasts for long periods of time. On the other hand, pigment is superior to dye in durability, but spectral characteristics such as transmission, absorption, and reflection change significantly because, on the contrary to dye whose coloring owes to a molecule, coloring by pigment owes to a solid (crystal) so that light scattering cannot be ignored depending on size of particles thereof. Especially, a yellow pigment is often inferior to other pigments such as a blue and a red pigment in color characteristics such as coloring power.

In a yellow color, it is required as its spectral characteristics that light of a short wavelength region (ca. 380 nm to ca. 500 nm) in the visible region of a wavelength region of 380 to 780 nm be absorbed and light of the rest of the wavelength region be transmitted or reflected. One example of the required spectral characteristics of water-soluble yellow dye in a color filter ink is that, as shown in Patent Document 1, transmittance of the aqueous solution thereof which is prepared so as to have transmittance of 20.0% at 435 nm be 95% or more at 610 nm. In patent Document 2, in a color filter for a reflective liquid crystal display using dye as a coloring material which contains each of yellow, magenta, and cyan pixel, the required spectral characteristics thereof are that the minimum value of the transmittance in a wavelength region of 420 nm to 470 nm be set between 4% to 40% on one pass of light through a yellow pixel while the maximum transmittance in a wavelength region of 500 nm to 700 nm be 80% or more. In the case of the dye as described in Patent Document 1 and Patent Document 2, almost ideal spectral characteristics can be readily obtained even when it is used as an ink; but in the case of the pigment, as mentioned above, it was difficult to satisfy the above-mentioned requirements. Accordingly, in many industry fields such as a coating material, an ink-jet ink, a color filter, and a toner, a yellow pigment composition having spectral characteristics equivalent to the required spectral characteristics of the yellow color as mentioned above has been wanted along with a method for producing the same.

One example to solve the problems mentioned above is to make pigment particles microparticlated. Transmittance and coloring power can be improved by making particle size of pigment fine to a level where light scattering can be ignored. To make microparticles, reported are so-called a solvent milling method and a solvent salt milling method, in which treatment with beads or an inorganic salt is done, such as those described in Patent Document 3.

However, in the solvent milling method and the solvent salt milling method, crystal growth and crushing of crystals occur in parallel, so that there have been problems of not only requiring large energy but also not expressing characteristics expected as pigment nanoparticles, such as color tone, transparency, spectral characteristics, and durability, because a strong force is applied to a pigment.

As shown in Patent Document 4, the applicant of the present invention proposed a novel method to produce pigment nanoparticles by separating pigments between processing surfaces being capable of approaching to and separating from; but a specific method for producing nanoparticles of a yellow pigment and its color characteristics were not disclosed therein.
Patent Document 1: Japanese Patent Laid-Open Publication No. H09-71744
Patent Document 2: Japanese Patent Laid-Open Publication No. H10-170716
Patent Document 3: Japanese Patent Laid-Open Publication No. 2004-292785
Patent Document 4: International Patent Laid-Open Publication No. 2009/008388

### DISCLOSURE OF INVENTION

### Problems To Be Solved By The Invention

In view of the situation mentioned above, the present invention has an object to provide; a yellow pigment composition which contains yellow pigment microparticles having spectral characteristics equivalent to the required spectral characteristics of the yellow color as mentioned above; and a method for producing the yellow pigment microparticles.

### Means For Solving The Problems

According to the present invention, provided is a yellow pigment composition containing at least one kind of yellow pigment microparticle characterized in that difference (Tmax−Tmin) between the maximum transmittance (Tmax) and the minimum transmittance (Tmin) of a transmission spectrum thereof in a region of 350 nm to 800 nm is 80% or more.

An embodiment of the present invention can be carried out as the yellow pigment microparticle, characterized in that the yellow pigment microparticle is an organic pigment.

Further, an embodiment of the present invention can be carried out as the yellow pigment microparticle, characterized in that the yellow pigment microparticle is an azo pigment or an isoindoline pigment.

Further, an embodiment of the present invention can be carried out as the yellow pigment microparticle, characterized in that the yellow pigment microparticle is formed by a process comprising:

a fluid to be processed is supplied between processing surfaces being capable of approaching to and separating from each other and displacing relative to each other,

pressure of force to move in the direction of approaching, including supply pressure of the fluid to be processed and pressure applied between the rotating processing surfaces, is balanced with pressure of force to move in the direction of separation thereby keeping a minute space in a distance between the processing surfaces,

the minute space kept between two processing surfaces is used as a flow path of the fluid to be processed, thereby forming a thin film fluid of the fluid to be processed, and

the microparticle is formed in this thin film fluid.

Further, an embodiment of the present invention can be carried out as the yellow pigment microparticle, characterized in that form of the yellow pigment microparticle is almost spherical.

Further, an embodiment of the present invention can be carried out as the yellow pigment microparticle, characterized in that a volume-average particle diameter of the yellow pigment microparticle is in the range of 1 nm to 200 nm.

According to the present invention, provided is a method to produce yellow pigment microparticles, a method to produce the yellow pigment microparticles, characterized in that:

a fluid to be processed is supplied between processing surfaces being capable of approaching to and separating from each other and displacing relative to each other,

pressure of force to move in the direction of approaching, including supply pressure of the fluid to be processed and pressure applied between the rotating processing surfaces, is balanced with pressure of force to move in the direction of separation thereby keeping a minute space in the distance between the processing surfaces,

the minute space kept between two processing surfaces is used as a flow path of the fluid to be processed, thereby forming a thin film fluid of the fluid to be processed, and

the yellow pigment microparticles are separated in this thin film fluid.

Further, an embodiment of the present invention can be carried out as a method for producing yellow pigment microparticles, characterized in that the method comprises:

a fluid pressure imparting mechanism for imparting pressure to a fluid to be processed,

at least two processing members of a first processing member and a second processing member, the second processing member being capable of relatively approaching to and separating from the first processing member, and

a rotation drive mechanism for rotating the first processing member and the second processing member relative to each other; wherein

each of the processing members is provided with at least two processing surfaces of a first processing surface and a second processing surface disposed in a position they are faced with each other,

each of the processing surfaces constitutes part of a forced flow path through which the fluid to be processed under the pressure is passed,

of the first and second processing members, at least the second processing member is provided with a pressure-receiving surface, and at least part of the pressure-receiving surface is comprised of the second processing surface,

the pressure-receiving surface receives pressure applied to the fluid to be processed by the fluid pressure imparting mechanism thereby generating force to move in the direction of separating the second processing surface from the first processing surface,

the fluid to be processed under the pressure is passed between the first and second processing surfaces being capable of approaching to and separating from each other and rotating relative to each other, whereby the fluid to be processed forms the thin film fluid, and

the yellow pigment microparticles are separated in this thin film fluid.

Further, an embodiment of the present invention can be carried out as a method for producing yellow pigment microparticles, characterized in that:

one kind of fluid to be processed is introduced to between the first processing surface and the second processing surface,

an another independent introduction path for another kind of fluid to be processed other than the one kind of the fluid to be processed is provided,

at least one opening leading to this introduction path is arranged in at least either one of the first processing surface or the second processing surface,

the another kind of the fluid to be processed is introduced between both the processing surfaces through this introduction path, and

the one kind of the fluid to be processed and the another kind of the fluid to be processed are mixed in the thin film fluid.

Further, an embodiment of the present invention can be carried out as a method for producing yellow pigment microparticles, characterized in that:

the opening is arranged in a downstream side of a point at which the one kind of the fluid to be processed becomes a laminar flow between both the processing surfaces, and

mixing of the fluids to be processed is done by introducing the another kind of the fluid to be processed from the opening.

### Advantages

According to the present invention, what could be provided are: a yellow pigment composition containing at least one kind of yellow pigment microparticle, characterized in that difference (Tmax−Tmin) between the maximum transmittance (Tmax) and the minimum transmittance (Tmin) of a transmission spectrum thereof in the region of 350 nm to 800 nm is 80% or more; and a method for producing the said yellow pigment microparticles. The yellow pigment composition as mentioned above has spectral characteristics almost equivalent to the required spectral characteristics of the yellow color described in Patent Document 1 and Patent Document 2, so that existing problems as mentioned before could be remedied.

### BRIEF DESCRIPTION OF DRAWINGS

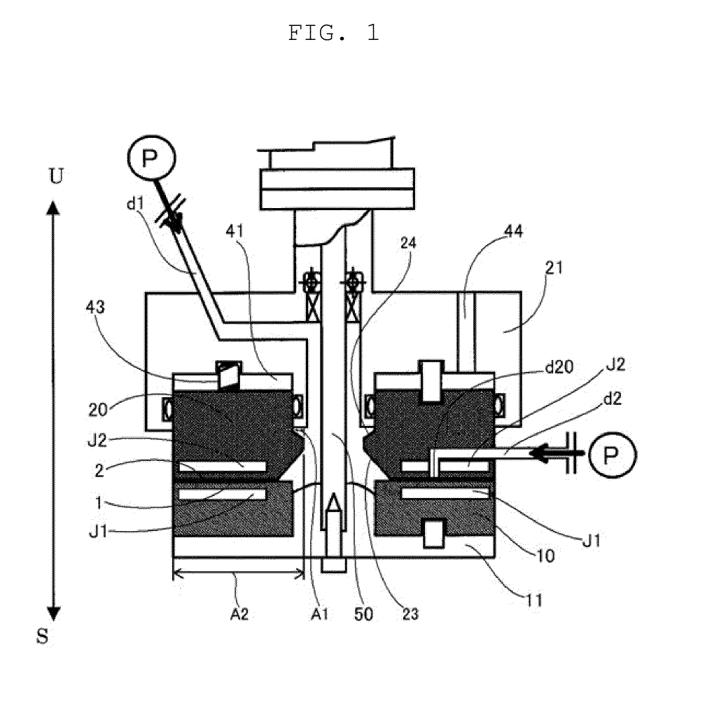

### [FIG. 1]

FIG. 1 is a schematic sectional view showing the fluid processing apparatus according to an embodiment of the present invention.

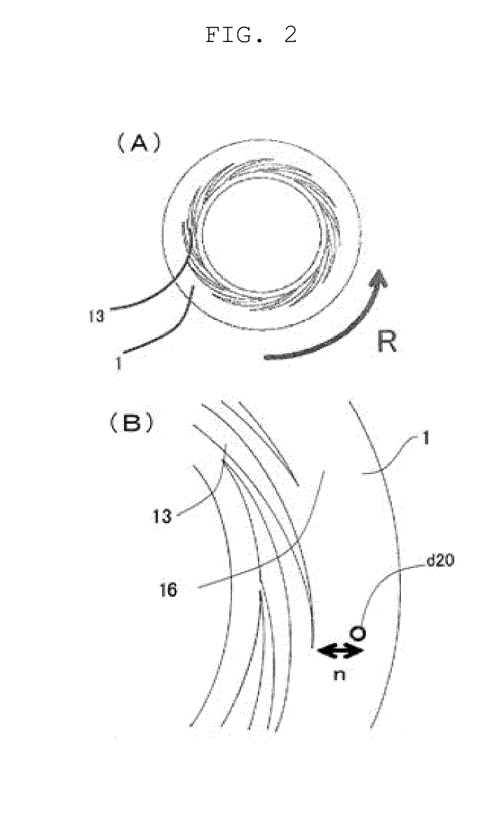

### [FIG. 2]

FIG. 2 (A) is a schematic plane view of the first processing surface in the fluid processing apparatus shown in FIG. 1, and FIG. 2 (B) is an enlarged view showing an important part of the processing surface in the apparatus.

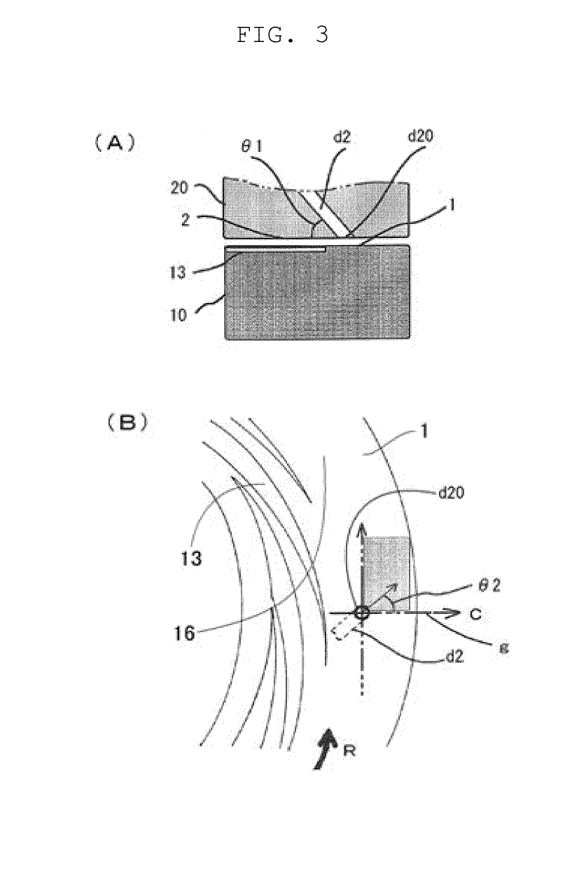

### [FIG. 3]

FIG. 3 (A) is a sectional view of the second introduction part of the apparatus, and FIG. 3 (B) is an enlarged view showing an important part of the processing surface for explaining the second introduction part.

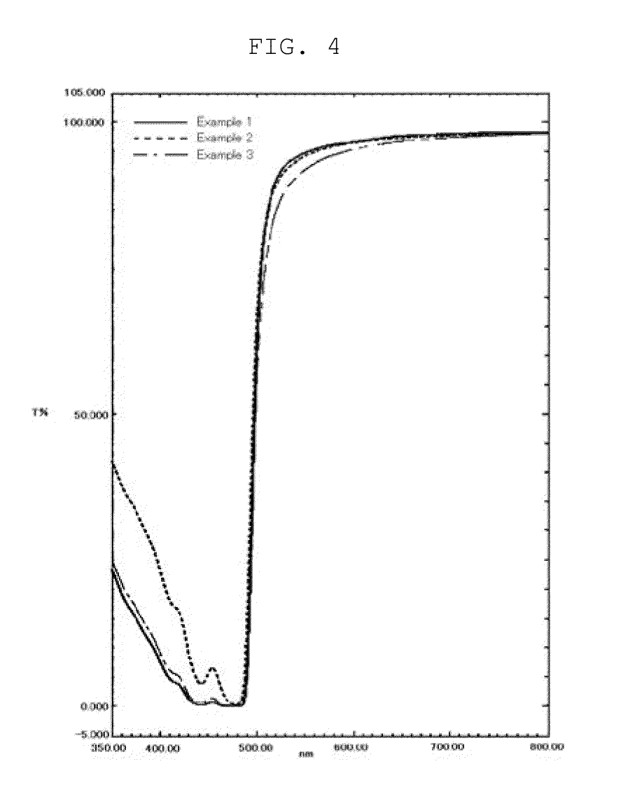

### [FIG. 4]

FIG. 4 shows transmission spectra of dispersion solutions of PY-185 microparticles (PY-185 concentration of 0.003% by weight) prepared in Example 1 (solid line), Example 2 (dashed line), and Example 3 (dashed-dotted line) of the present invention.

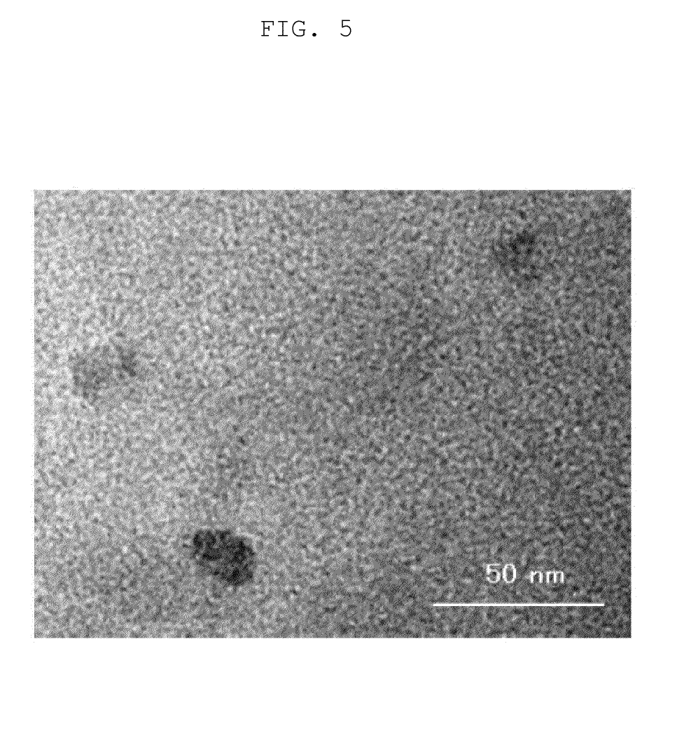

### [FIG. 5]

FIG. 5 shows a TEM picture of the PY-185 microparticles prepared in Example 1 of the present invention.

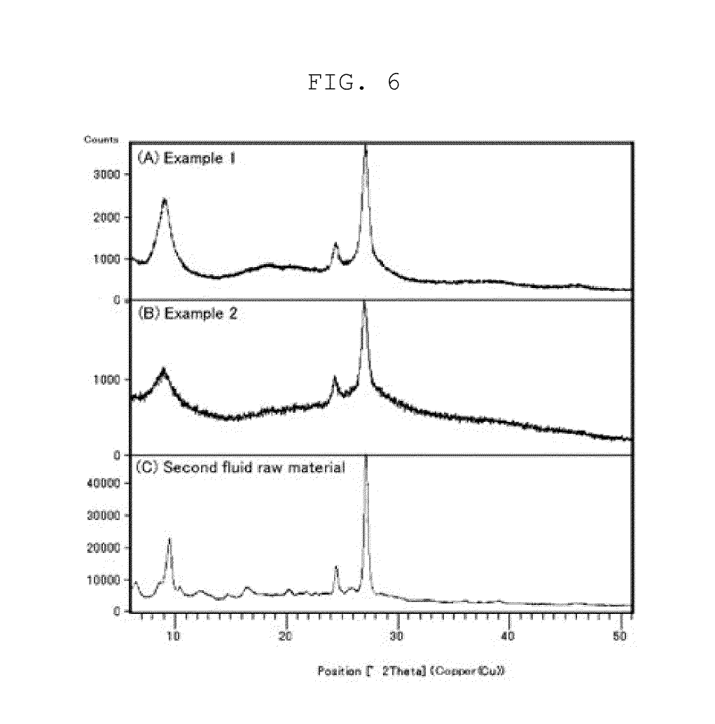

### [FIG. 6]

FIG. 6 shows powder X-ray diffraction spectrum charts of (A) PY-185 microparticles prepared in Example 1, (B) PY-185 microparticles prepared in Example 2, and (C) PY-185 used as a starting raw material.

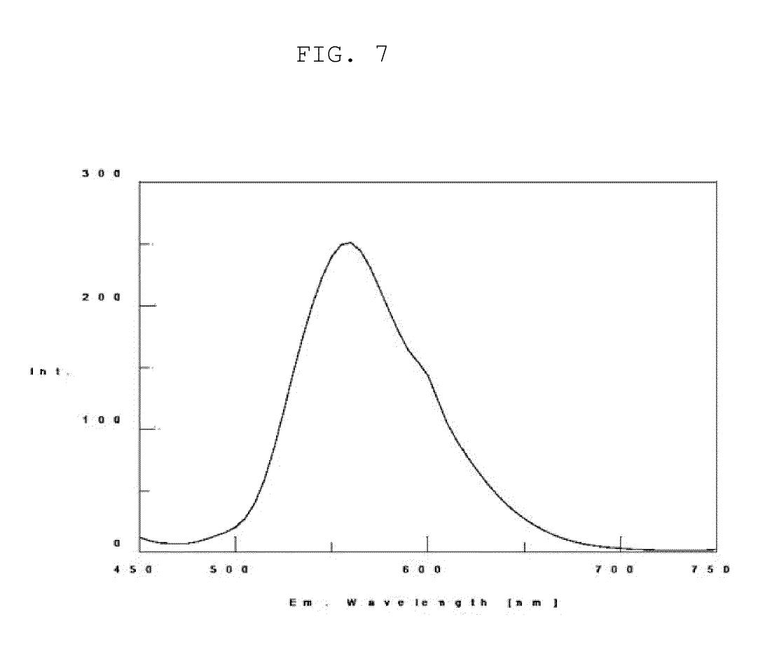

### [FIG. 7]

FIG. 7 shows fluorescence spectrum of the PY-185 microparticle powders prepared in Example 1 with exciting wavelength of 400 nm.

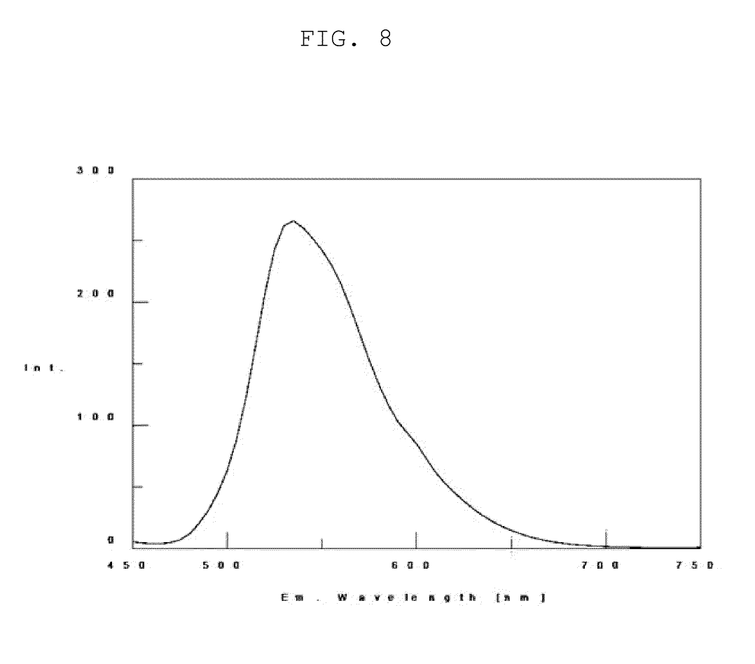

### [FIG. 8]

FIG. 8 shows fluorescence spectrum of the PY-185 powders used as a starting raw material with exciting wavelength of 400 nm.

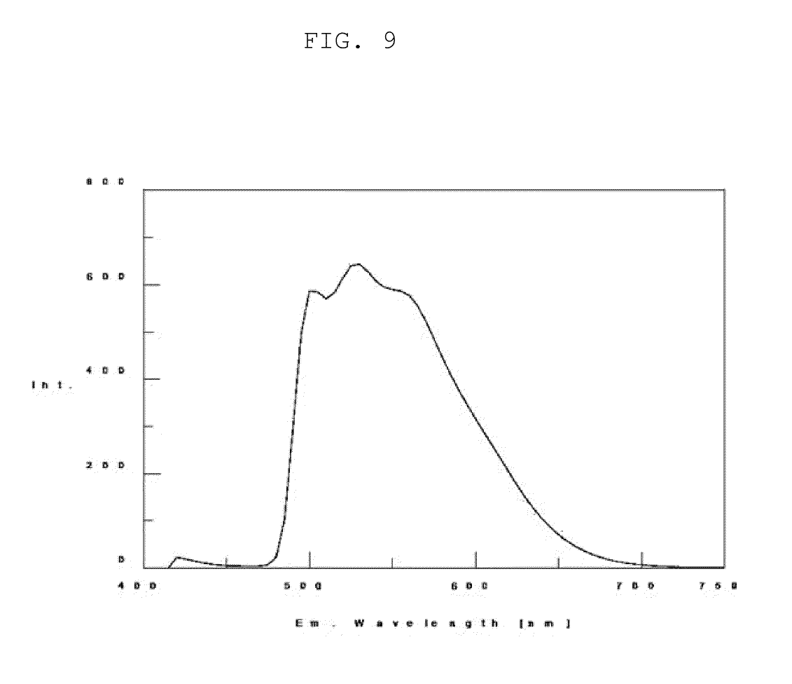

### [FIG. 9]

FIG. 9 shows fluorescence spectrum of dispersion solution of the PY-185 microparticles prepared in Example 1 (pigment concentration of 0.003% by weight) with exciting wavelength of 400 nm.

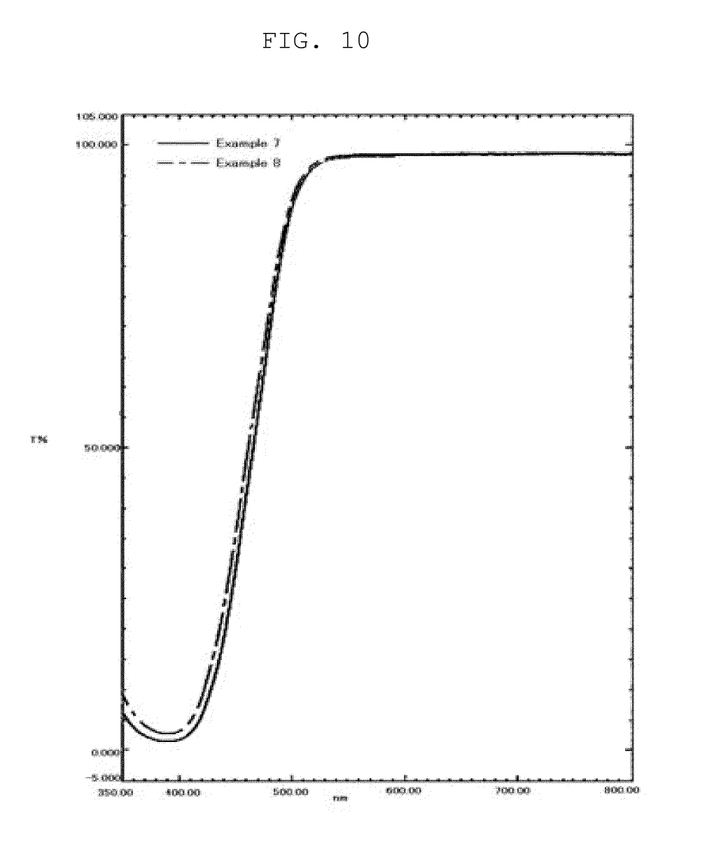

### [FIG. 10]

FIG. 10 shows transmission spectra of dispersion solutions of PY-155 microparticles (PY-155 concentration of 0.004% by weight) prepared in Example 7 (solid line) and Example 8 (dashed-dotted line) of the present invention.

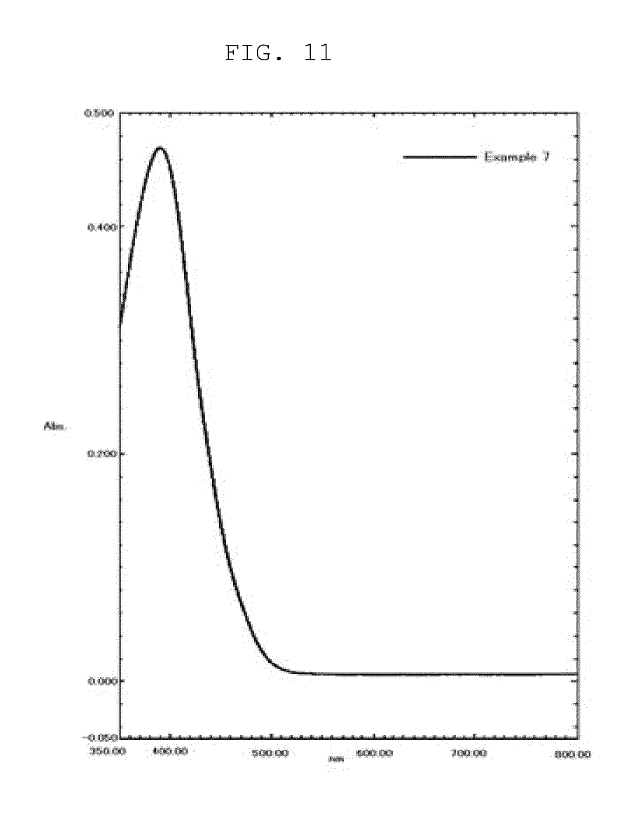

### [FIG. 11]

FIG. 11 shows absorption spectrum of the dispersion solution of the PY-155 microparticles (PY-155 concentration of 0.0014% by weight) prepared in Example 7 of the present invention.

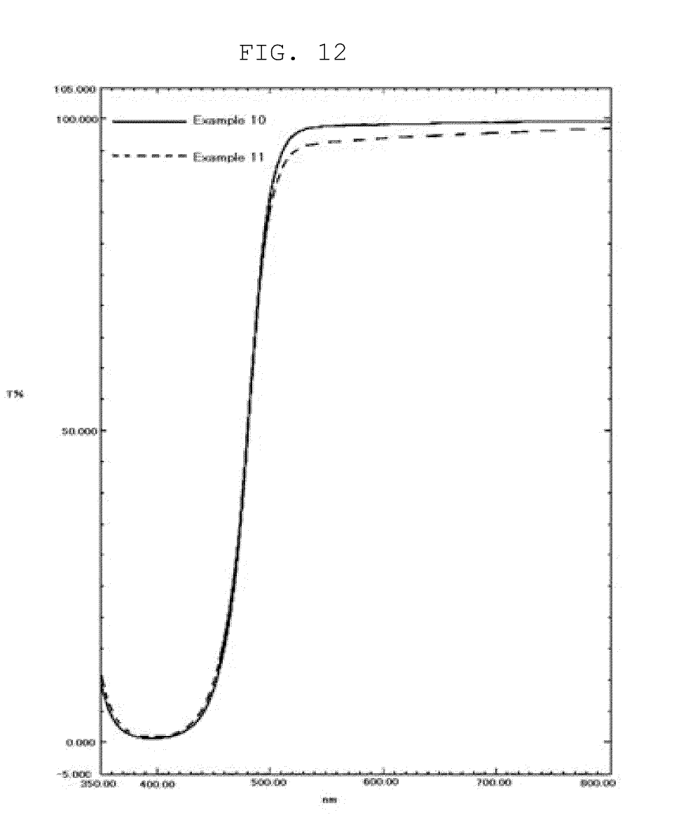

### [FIG. 12]

FIG. 12 shows transmission spectra of dispersion solutions of PY-180 microparticles (PY-180 concentration of 0.004% by weight) prepared in Example 10 (solid line) and Example 11 (dashed line) of the present invention.

### MODES FOR CARRYING OUT THE INVENTION

The yellow pigment microparticles to constitute the yellow pigment composition of the present invention is not particularly restricted; and an illustrative example of the yellow pigment microparticles includes an organic pigment of an azo pigment such as a disazo yellow pigment, a monoazo yellow pigment, an azo-lake yellow pigment, and a condensed azo yellow pigment; an organic pigment such as an isoindolinone yellow pigment, an isoindoline yellow pigment, a condensed polycylic yellow pigment, and a quinacridone pigment; and an inorganic pigment such as a strontium yellow and a zinc yellow. Further, included therein are those in the color index name of C. I. Pigment Yellow 1, 2, 3, 4, 5, 6, 7, 9, 10, 12, 13, 14, 15, 16, 17, 24, 32, 34, 35, 35:1, 36, 36:1, 37, 37:1, 40, 42, 43, 53, 55, 60, 61, 62, 63, 65, 73, 74, 77, 81, 83, 86, 93, 94, 95, 97, 98, 100, 101, 104, 106, 108, 109, 110, 113, 114, 115, 116, 117, 118, 119, 120, 122, 123, 125, 126, 127, 128, 129, 137, 138, 139, 147, 148, 150, 151, 152, 153, 154, 155, 156, 161, 162, 164, 166, 167, 168, 169, 170, 171, 172, 173, 174, 175, 176, 177, 179, 180, 181, 182, 185, 187, 188, 193, 194, 199, 213, and 214, or derivatives of them. The yellow pigments mentioned above may be used singly or as a mixture of a plurality of them.

The present invention relates to a yellow pigment composition containing at least one kind of yellow pigment microparticle having any of the transmission spectra shown in FIG. 4, FIG. 10 and FIG. 12. In addition, the yellow pigment composition of the present invention includes a yellow derivative such as sulfonated and hydroxylated yellow pigment microparticles. Further, the present invention includes a yellow pigment composition having a surface of the yellow pigment microparticles introduced with a functional group such as a hydroxyl group and a sulfo group. In the yellow pigment composition of the present invention, there is no particular restriction as to a crystal type thereof.

As shown in the transmission spectra of FIG. 4, FIG. 10, or FIG. 12, it can be seen that the yellow pigment composition contains at least one kind of yellow pigment microparticle characterized in that difference (Tmax−Tmin) between the maximum transmittance (Tmax) and the minimum transmittance (Tmin) of the transmission spectrum thereof in the region of 350 nm to 800 nm is 80% or more. More preferably, the yellow pigment composition contains at least one kind of yellow pigment microparticle characterized in that difference (Tmax−Tmin) between the maximum transmittance (Tmax) and the minimum transmittance (Tmin) of the transmission spectrum thereof in the region of 350 nm to 800 nm is 80% or more and less than 100%. Measurement method of the transmission spectrum in the present invention is not particularly restricted. Therefore, the measurement method includes, for example, a method in which the transmission spectrum of the yellow pigment composition is measured as to its dispersion solution in an aqueous medium or in an organic solvent, and a method in which the measurements are done after the pigment is applied onto a glass, a transparent electrode, or a film.

A method for producing the yellow pigment composition obtained by the present invention is not particularly restricted. A build-up method as well as a break-down method represented by a crushing method may be used. Alternatively, it may be newly synthesized.

As one example of a method for producing the yellow pigment composition of the present invention, in the method for producing the yellow pigment microparticles by mixing a fluid which contains a yellow pigment solution having a yellow pigment dissolved in a solvent with a fluid which contains a solvent capable of being a poor solvent to a yellow pigment, whereby separating the yellow pigment, the method characterized in that each of the foregoing fluids are mixed in a thin film fluid formed between processing surfaces being capable of relatively approaching to and separating from each other and disposed in a position they are faced with each other, wherein at least one of the surfaces rotates relative to the other, thereby separating the yellow pigment microparticles in the thin film fluid may be used. Hereinafter, this producing method will be explained. However, this producing method is a mere one example, and thus, the present invention is not limited to this producing method.

A starting material yellow pigment to be dissolved in a solvent to prepare a yellow pigment solution is not particularly restricted; and thus, a yellow pigment which is the same kind as the yellow pigment microparticles to constitute the foregoing yellow pigment composition may be used. The yellow pigment mentioned above may be used singly or as a mixture of plurality of them to form a solid solution. Meanwhile, a crystal type of the yellow pigment before dissolving into the afore-mentioned solvent is not particularly restricted; and thus, various crystal types of yellow pigments may be used. In addition, a yellow pigment before a step to make it a pigment and a yellow pigment containing an amorphous yellow pigment may be used. A particle diameter thereof is not particularly restricted, either.

Hereinbelow, a fluid processing apparatus usable in this method will be explained.

The fluid processing apparatus shown in FIG. 1 to FIG. 3 is similar to the apparatus described in Patent Document 4, with which a material to be processed is processed between processing surfaces in processing members arranged so as to be able to approach to and separate from each other, at least one of which rotates relative to the other; wherein, of the fluids to be processed, a first fluid to be processed, i.e., a first fluid, is introduced into between the processing surfaces, and a second fluid to be processed, i.e., a second fluid, is introduced into between the processing surfaces from a separate path that is independent of the flow path introducing the afore-mentioned first fluid and has an opening leading to between the processing surfaces, whereby the first fluid and the second fluid are mixed and stirred between the processing surfaces. Meanwhile, in FIG. 1, a reference character U indicates an upside and a reference character S indicates a downside; however, up and down, frond and back and right and left shown therein indicate merely a relative positional relationship and does not indicate an absolute position. In FIG. 2 (A) and FIG. 3 (B), reference character R indicates a rotational direction. In FIG. 3 (C), reference character C indicates a direction of centrifugal force (a radial direction).

In this apparatus provided with processing surfaces arranged opposite to each other so as to be able to approach to and separate from each other, at least one of which rotates relative to the other, at least two kinds of fluids to be processed are used as the fluid to be processed, wherein at least one fluid thereof contains at least one kind of material to be processed, a thin film fluid is formed by converging the respective fluids between these processing surfaces, and the material to be processed is processed in this thin film fluid. With this apparatus, a plurality of fluids to be processed may be processed as mentioned above; but a single fluid to be processed may be processed as well.

This fluid processing apparatus is provided with two processing members of a first processing member 10 and a second processing member 20 arranged opposite to each other, wherein at least one of these processing members rotates. The surfaces arranged opposite to each other of the respective processing members 10 and 20 are made to be the respective processing surfaces. The first processing member 10 is provided with a first processing surface 1 and the second processing member 20 is provided with a second processing surface 2.

The processing surfaces 1 and 2 are connected to a flow path of the fluid to be processed and constitute part of the flow path of the fluid to be processed. Distance between these processing surfaces 1 and 2 can be changed as appropriate; and thus, the distance thereof is controlled so as to form a minute space usually less than 1 mm, for example, in the range of about 0.1 μm to about 50 μm. With this, the fluid to be processed passing through between the processing surfaces 1 and 2 becomes a forced thin film fluid forced by the processing surfaces 1 and 2.

When a plurality of fluids to be processed are processed by using this apparatus, the apparatus is connected to a flow path of the first fluid to be processed whereby forming part of the flow path of the first fluid to be processed; and part of the flow path of the second fluid to be processed other than the first fluid to be processed is formed. In this apparatus, the two paths converge into one, and two fluids to be processed are mixed between the processing surfaces 1 and 2 so that the fluids may be processed by reaction and so on. It is noted here that the term “process(ing)” includes not only the embodiment wherein a material to be processed is reacted but also the embodiment wherein a material to be processed is only mixed or dispersed without accompanying reaction.

To specifically explain, this apparatus is provided with a first holder 11 for holding the first processing member 10, a second holder 21 for holding the second processing member 20, a surface-approaching pressure imparting mechanism, a rotation drive member, a first introduction part d1, a second introduction part d2, and a fluid pressure imparting mechanism p.

As shown in FIG. 2 (A), in this embodiment, the first processing member 10 is a circular body, or more specifically a disk with a ring form. Similarly, the second processing member 20 is a disk with a ring form. A material of the processing members 10 and 20 is not only metal but also carbon, ceramics, sintered metal, abrasion-resistant steel, sapphire, other metal subjected to hardening treatment, and rigid material subjected to lining, coating, or plating. In the processing members 10 and 20 of this embodiment, at least part of the first and the second surfaces 1 and 2 arranged opposite to each other is mirror-polished.

Roughness of this mirror polished surface is not particularly limited; but surface roughness Ra is preferably 0.01 μm to 1.0 μm, or more preferably 0.03 μm to 0.3 μm.

At least one of the holders can rotate relative to the other holder by a rotation drive mechanism such as an electric motor (not shown in drawings). A reference numeral 50 in FIG. 1 indicates a rotary shaft of the rotation drive mechanism; in this embodiment, the first holder 11 attached to this rotary shaft 50 rotates, and thereby the first processing member 10 attached to this first holder 11 rotates relative to the second processing member 20. As a matter of course, the second processing member 20 maybe made to rotate, or the both may be made to rotate. Further in this embodiment, the first and second holders 11 and 21 may be fixed, while the first and second processing members 10 and 20 maybe made to rotate relative to the first and second holders 11 and 21.

At least any one of the first processing member 10 and the second processing member 20 is able to approach to and separate from at least any other member, thereby the processing surfaces 1 and 2 are able to approach to and separate from each other.

In this embodiment, the second processing member 20 approaches to and separates from the first processing member 10, wherein the second processing member 20 is accepted in an accepting part 41 arranged in the second holder 21 so as to be able to rise and set. However, as opposed to the above, the first processing member 10 may approach to and separate from the second processing member 20, or both of the processing members 10 and 20 may approach to and separate from each other.

This accepting part 41 is a concave portion for mainly accepting that side of the second processing member 20 opposite to the second processing surface 2, and this concave portion is a groove being formed into a circle, i.e., a ring when viewed in a plane. This accepting part 41 accepts the second processing member 20 with sufficient clearance so that the second processing member 20 may rotate. Meanwhile, the second processing member 20 may be arranged so as to be movable only parallel to the axial direction; alternatively, the second processing member 20 may be made movable, by making this clearance larger, relative to the accepting part 41 so as to make the center line of the processing member 20 inclined, namely unparallel, to the axial direction of the accepting part 41, or movable so as to deviate the center line of the processing member 20 and the center line of the accepting part 41 toward the radius direction.

It is preferable that the second processing member 20 be accepted by a floating mechanism so as to be movable in the three dimensional direction, as described above.

The fluids to be processed are introduced into between the processing surfaces 1 and 2 from the first introduction part d1 and the second introduction part d2 under the state that pressure is applied thereto by a fluid pressure imparting mechanism p consisting of various pumps, potential energy, and so on. In this embodiment, the first introduction part d1 is a flow path arranged in the center of the circular second holder 21, and one end thereof is introduced into between the processing surfaces 1 and 2 from inside the circular processing members 10 and 20. Through the second introduction part d2, the second fluid to be processed for reaction to the first fluid to be processed is introduced into between the processing surfaces 1 and 2. In this embodiment, the second introduction part d2 is a flow path arranged inside the second processing member 20, and one end thereof is open at the second processing surface 2 . The first fluid to be processed which is pressurized with the fluid pressure imparting mechanism p is introduced from the first introduction part d1 to the space inside the processing members 10 and 20 so as to pass through between the first and second processing surfaces 1 and 2 to outside the processing members 10 and 20. From the second introduction part d2, the second fluid to be processed which is pressurized with the fluid pressure imparting mechanism p is provided into between the processing surfaces 1 and 2, whereat this fluid is converged with the first fluid to be processed, and there, various fluid processing such as mixing, stirring, emulsification, dispersion, reaction, deposition, crystallization, and separation are effected, and then the fluid thus processed is discharged from the processing surfaces 1 and 2 to outside the processing members 10 and 20. Meanwhile, an environment outside the processing members 10 and 20 may be made negative pressure by a vacuum pump.

The surface-approaching pressure imparting mechanism mentioned above supplies the processing members with force exerting in the direction of approaching the first processing surface 1 and the second processing surface 2 each other. In this embodiment, the surface-approaching pressure imparting mechanism is arranged in the second holder 21 and biases the second processing member 20 toward the first processing member 10.

The surface-approaching pressure imparting mechanism is a mechanism to generate a force (hereinafter “surface-approaching pressure”) to press the first processing surface 1 of the first processing member 10 and the second processing surface 2 of the second processing member 20 in the direction to make them approach to each other. By the balance between this surface-approaching pressure and the force to separate the processing surfaces 1 and 2 from each other, i.e., the force such as the fluid pressure, a thin film fluid having minute thickness in a level of nanometer or micrometer is generated. In other words, the distance between the processing surfaces 1 and 2 is kept in a predetermined minute distance by the balance between these forces.

In the embodiment shown in FIG. 1, the surface-approaching pressure imparting mechanism is arranged between the accepting part 41 and the second processing member 20. Specifically, the surface-approaching pressure imparting mechanism is composed of a spring 43 to bias the second processing member 20 toward the first processing member 10 and a biasing-fluid introduction part 44 to introduce a biasing fluid such as air and oil, wherein the surface-approaching pressure is provided by the spring 43 and the fluid pressure of the biasing fluid. The surface-approaching pressure may be provided by any one of this spring 43 and the fluid pressure of this biasing fluid; and other forces such as magnetic force and gravitation may also be used. The second processing member 20 recedes from the first processing member 10 thereby making a minute space between the processing surfaces by separating force, caused by viscosity and the pressure of the fluid to be processed applied by the fluid pressure imparting mechanism p, against the bias of this surface-approaching pressure imparting mechanism. By this balance between the surface-approaching pressure and the separating force as mentioned above, the first processing surface 1 and the second processing surface 2 can be set with the precision of a micrometer level; and thus the minute space between the processing surfaces 1 and 2 may be set. The separating force mentioned above includes fluid pressure and viscosity of the fluid to be processed, centrifugal force by rotation of the processing members, negative pressure when negative pressure is applied to the biasing-fluid introduction part 44, and spring force when the spring 43 works as a pulling spring. This surface-approaching pressure imparting mechanism may be arranged also in the first processing member 10, in place of the second processing member 20, or in both of the processing members.

To specifically explain the separation force, the second processing member 20 has the second processing surface 2 and a separation controlling surface 23 which is positioned inside the processing surface 2 (namely at the entering side of the fluid to be processed into between the first and second processing surfaces 1 and 2) and next to the second processing surface 2. In this embodiment, the separation controlling surface 23 is an inclined plane, but may be a horizontal plane. The pressure of the fluid to be processed acts to the separation controlling surface 23 to generate force directing to separate the second processing member 20 from the first processing member 10. Therefore, the second processing surface 2 and the separation controlling surface 23 constitute a pressure receiving surface to generate the separation force.

In the example shown in FIG. 1, an approach controlling surface 24 is formed in the second processing member 20. This approach controlling surface 24 is a plane opposite, in the axial direction, to the separation controlling surface 23 (upper plane in FIG. 1) and, by action of pressure applied to the fluid to be processed, generates force of approaching the second processing member 20 toward the first processing member 10.

Meanwhile, the pressure of the fluid to be processed exerted on the second processing surface 2 and the separation controlling surface 23, i.e., the fluid pressure, is understood as force constituting an opening force in a mechanical seal. The ratio (area ratio A1/A2) of a projected area A1 of the approach controlling surface 24 projected on a virtual plane perpendicular to the direction of approaching and separating the processing surfaces 1 and 2, that is, to the direction of rising and setting of the second processing member 20 (axial direction in FIG. 1), to a total area A2 of the projected area of the second processing surface 2 of the second processing member 20 and the separation controlling surface 23 projected on the virtual plane is called as balance ratio K, which is important for control of the opening force. This opening force can be controlled by the pressure of the fluid to be processed, i.e., the fluid pressure, by changing the balance line, i.e., by changing the area A1 of the approach controlling surface 24.

Sliding surface actual surface pressure P, i.e., the fluid pressure out of the surface-approaching pressures, is calculated according to the following equation:

P=P1×(K−k)+Ps

Here, P1 represents the pressure of a fluid to be processed, i.e., the fluid pressure, K represents the balance ratio, k represents an opening force coefficient, and Ps represents a spring and back pressure.

By controlling this balance line to control the sliding surface actual surface pressure P, the space between the processing surfaces 1 and 2 is formed as a desired minute space, thereby forming a fluid film of the fluid to be processed so as to make the processed substance such as a product fine and to effect uniform processing by reaction.

Meanwhile, the approach controlling surface 24 may have a larger area than the separation controlling surface 23, though this is not shown in the drawing.

The fluid to be processed becomes a forced thin film fluid by the processing surfaces 1 and 2 that keep the minute space therebetween, whereby the fluid is forced to move out from the circular, processing surfaces 1 and 2. However, the first processing member 10 is rotating; and thus, the mixed fluid to be processed does not move linearly from inside the circular, processing surfaces 1 and 2 to outside thereof, but does move spirally from the inside to the outside thereof by a resultant vector acting on the fluid to be processed, the vector being composed of a moving vector toward the radius direction of the circle and a moving vector toward the circumferential direction.

Meanwhile, a rotary shaft 50 is not only limited to be placed vertically, but may also be placed horizontally, or at a slant. This is because the fluid to be processed is processed in a minute space between the processing surfaces 1 and 2 so that the influence of gravity can be substantially eliminated. In addition, this surface-approaching pressure imparting mechanism can function as a buffer mechanism of micro-vibration and rotation alignment by concurrent use of the foregoing floating mechanism with which the second processing member 20 may be held displaceably.

In the first and second processing members 10 and 20, the temperature thereof may be controlled by cooling or heating at least any one of them; in FIG. 1, an embodiment having temperature regulating mechanisms J1 and J2 in the first and second processing members 10 and 20 is shown. Alternatively, the temperature may be regulated by cooling or heating the introducing fluid to be processed. These temperatures may be used to separate the processed substance or may be set so as to generate Benard convection or Marangoni convection in the fluid to be processed between the first and second processing surfaces 1 and 2.

As shown in FIG. 2, in the first processing surface 1 of the first processing member 10, a groove-like depression 13 extended toward an outer side from the central part of the first processing member 10, namely in a radius direction, may be formed. The depression 13 may be, as a plane view, curved or spirally extended on the first processing surface 1 as shown in FIG. 2 (B), or, though not shown in the drawing, maybe extended straight radially, or bent at a right angle, or jogged; and the depression may be continuous, intermittent, or branched. In addition, this depression 13 may be formed also on the second processing surface 2, or on both of the first and second processing surfaces 1 and 2. By forming the depression 13 as mentioned above, the micro-pump effect can be obtained so that the fluid to be processed may be sucked into between the first and second processing surfaces 10 and 20.

The base end of the depression 13 reaches preferably inner circumference of the first processing member 10. The front end of the depression 13 extends in an outer circumferential direction of the first processing surface 1 with the depth thereof (cross-sectional area) being gradually shallower as going from the base end toward the front end.

Between the front end of the depression 13 and the outer periphery of the first processing surface 1 is arranged a flat surface 16 not having the depression 13.

When an opening d20 of the second introduction part d2 is arranged in the second processing surface 2, the arrangement is done preferably at a position opposite to the flat surface 16 of the first processing surface 1 arranged at a position opposite thereto.

This opening d20 is arranged preferably in the downstream (outside in this case) of the depression 13 of the first processing surface 1. The opening is arranged especially preferably at a position opposite to the flat surface 16 located nearer to the outer diameter than a position where the direction of flow upon introduction by the micro-pump effect is changed to the direction of a spiral and laminar flow formed between the processing surfaces. Specifically, in FIG. 2 (B), a distance n from the outermost side of the depression 13 arranged in the first processing surface 1 in the radial direction is preferably about 0.5 mm or more. Especially in the case of separating nanosized microparticles (nanoparticles) from a fluid, it is preferable that mixing of a plurality of fluids to be processed and separation of the nanoparticles therefrom be effected under the condition of a laminar flow.

This second introduction part d2 may have directionality. For example, as shown in FIG. 3 (A), the direction of introduction from the opening d20 of the second processing surface 2 is inclined at a predetermined elevation angle (θ1) relative to the second processing surface 2. The elevation angle (θ1) is set at more than 0° and less than 90°, and when the reaction speed is high, the angle (θ1) is preferably set in the range of 1° to 45°.

In addition, as shown in FIG. 3 (B), introduction from the opening d20 of the second processing surface 2 has directionality in a plane along the second processing surface 2. The direction of introduction of this second fluid is in the outward direction departing from the center in a radial component of the processing surface and in the forward direction in a rotation component of the fluid between the rotating processing surfaces. In other words, a predetermined angle (θ2) exists facing the rotation direction R from a reference line g, which is the line to the outward direction and in the radial direction passing through the opening d20. This angle (θ2) is also set preferably at more than 0° and less than 90°.

This angle (θ2) can vary depending on various conditions such as the type of fluid, the reaction speed, viscosity, and the rotation speed of the processing surface. In addition, it is also possible not to give the directionality to the second introduction part d2 at all.

In the embodiment shown in FIG. 1, kinds of the fluid to be processed and numbers of the flow path thereof are set two respectively; but they may be one, or three or more. In the embodiment shown in FIG. 1, the second fluid is introduced into between the processing surfaces 1 and 2 from the introduction part d2; but this introduction part may be arranged in the first processing member 10 or in both. Alternatively, a plurality of introduction parts may be arranged relative to one fluid to be processed. The opening for introduction arranged in each processing member is not particularly restricted in its form, size, and number; and these may be changed as appropriate. The opening of the introduction part may be arranged just before the first and second processing surfaces 1 and 2 or in the side of further upstream thereof.

In the apparatus mentioned above, treatment such as separation and deposition, or crystallization takes place under a forced and uniform mixing between the processing surfaces 1 and 2 arranged opposite to each other so as to be able to approach to and separate from each other, at least one of which rotates relative to the other, as shown in FIG. 1. A particle diameter and mono-dispersibility of the yellow pigment microparticles can be controlled by appropriately controlling rotation number of the processing members 10 and 20, fluid velocity, distance between the processing surfaces, raw material concentration of a fluid to be processed, solvent species of a fluid to be processed, and so on.

Hereinafter, the reaction of production of yellow pigment microparticles in the present invention is described in more detail.

First, a fluid containing a solvent capable of being a poor solvent to a yellow pigment is introduced as a first fluid through one flow path, that is, the first introduction part d1, into the space between the processing surfaces 1 and 2 arranged to be opposite to each other so as to be able to approach to and separate from each other, at least one of which rotates relative to the other, thereby forming a thin film fluid comprised of the first fluid between the processing surfaces.

Then, from the second introduction part d2 which is a separate flow path, as the second fluid, a fluid containing a yellow pigment solution having a yellow pigment (this is a reaction material) dissolved is directly introduced into the thin film fluid formed by the first fluid. Meanwhile, of the first fluid and the second fluid, in at least any one of them is contained an organic solvent generally capable of transforming a crystal type of a copper phthalocyanine to other than the α-type crystal (this solvent will be mentioned later).

As described above, the first fluid and the second fluid are instantly mixed with maintaining a state of a ultrathin film between the processing surfaces 1 and 2, the distance of which is regulated by the pressure balance between the supply pressure of the fluids and the pressure exerted between the rotating processing surfaces, thereby enabling to carryout the reaction producing the yellow pigment microparticles.

To effect the reaction between the processing surfaces 1 and 2, the second fluid may be introduced through the first introduction part d1 and the first fluid through the second introduction part d2, as opposed to the above description. That is, the expression “first” or “second” for each solvent has a meaning for merely discriminating an n^{th} solvent among a plurality of solvents present, and third or more solvents can also be present.

As mentioned above, the third introduction part d3, in addition to the first introduction part d1 and the second introduction part d2, may also be arranged in the processing apparatus, so that the first fluid, the second fluid, and the third fluid may be introduced separately into the processing apparatus through the respective introduction parts. By so doing, concentration and pressure of each solution can be controlled separately so that the separation reaction and stabilization of a particle diameter of the yellow pigment microparticles can be controlled more precisely. Meanwhile, a combination of fluids to be processed (first to third fluids) that are introduced into respective introduction parts may be arbitrarily chosen. So are the cases of arranging introduction parts of the fourth introduction part or more, whereby the fluids to be introduced into the processing apparatus can be fragmented. In addition, temperatures of the fluids to be processed, i.e., the first fluid, the second fluid, and so on, may be controlled; and temperature difference among the first fluid, the second fluid, and so on (namely, temperature difference among each of the introducing fluids to be processed) may be controlled either. To control temperature and temperature difference of each of the introducing fluids to be processed, a mechanism to measure temperatures of each fluid to be processed (temperatures of the fluids in the processing apparatus, or more precisely just before introduction between the processing surfaces 1 and 2), by which the fluids to be processed that are introduced between the processing surfaces 1 and 2 can be heated or cooled, may be added to the apparatus.

Combination of the first fluid and the second fluid is not particularly restricted; a fluid which contains a yellow pigment solution having a yellow pigment dissolved in a solvent and a fluid which contains a solvent capable of being a poor solvent to the yellow pigment may be used. The solvent capable of being a poor solvent to a yellow pigment is defined as that this solvent is capable of being a poor solvent which has lower solubility to a yellow pigment than the solvent of the yellow pigment solution in which a yellow pigment is dissolved.

For example, a solvent for dissolving a yellow pigment is not particularly limited, and in the case of an acidic aqueous solution, for example, sulfuric acid, hydrochloric acid, nitric acid, trifluoroacetic acid, phosphoric acid, or citric acid can be used. Further, amide solvents such as 1-methyl-2-pyrrolidinone, 1,3-dimethyl-2-imidazolidinone, 2-pyrrolidinone, ε-caprolactam, formamide, N-methylformamide, N,N-dimethylformamide, acetamide, N-methylacetamide, N,N-dimethylacetamide, N-methylpropanamide, and hexamethyl phosphoric triamide; dimethyl sulfoxide; pyridine; or thier mixture can be used. In addition, a solution having a yellow pigment dissolved into a general organic solvent including the above-mentioned amide solvents, dimethyl sulfoxide, and pyridine that are added with an alkaline or an acidic substance may be used as a yellow pigment solution. An alkaline substance which is added to the organic solvent includes sodium hydroxide, potassium hydroxide, sodium methoxide, and sodium ethoxide, or the like. An acid substance, as the same described above, includes sulfuric acid, hydrochloric acid, nitric acid, trifluoroacetic acid, phosphoric acid, citric acid, or the like.

As to the solvent capable of being a poor solvent to separate yellow pigment microparticles, a solvent having lower solubility to a yellow pigment than the solvent into which a yellow pigment has been dissolved can be used. An illustrative example of the solvent like this includes water, an alcohol compound solvent, an amide compound solvent, a ketone compound solvent, an ether compound solvent, an aromatic compound solvent, carbon disulfide, an aliphatic compound solvent, a nitrile compound solvent, a sulfoxide compound solvent, a halogenated compound solvent, an ester compound solvent, a pyridine compound solvent, an ionic liquid solvent, a carboxylic acid compound solvent, a sulfonic acid compound solvent, and a sulfolane compound solvent. These solvents may be used singly or as a mixture of two or more of them. Further, a solvent, capable of being a poor solvent to a yellow pigment, to which an alkaline or an acidic substance is added may be used. An alkaline substance which is added to the solvent capable of being a poor solvent to a yellow solvent includes sodium hydroxide, potassium hydroxide, sodium methoxide, and sodium ethoxide, or the like, and an acid substance includes sulfuric acid, hydrochloric acid, nitric acid, trifluoroacetic acid, phosphoric acid, citric acid, or the like.

In addition, a dispersing agent such as a block copolymer, a macromolecular polymer, and a surfactant may be contained in any one of the fluid which contains a yellow pigment solution and the fluid which contains a solvent capable of being a poor solvent to a yellow pigment, or both fluids. Further, the foregoing dispersing agent may be contained in a third fluid which is different from any of the fluid which contains a yellow pigment solution and the fluid which contains a solvent capable of being a poor solvent to a yellow pigment.

As surfactants and dispersants, various commercial products for use in dispersing pigments can be used. The surfactants and dispersants include, but are not limited to, those based on dodecylbenzenesulfonic acid such as sodium dodecyl sulfate or Neogen R-K (Dai-ichi Kogyo Seiyaku Co., Ltd.), Solsperse 20000, Solsperse 24000, Solsperse 26000, Solsperse 27000, Solsperse 28000, and Solsperse 41090 (produced by Avecia Corporation), Disperbyk-160, Disperbyk-161, Disperbyk-162,Disperbyk-163, Disperbyk-166, Disperbyk-170, Disperbyk-180, Disperbyk-181, Disperbyk-182, Disperbyk-183, Disperbyk-184, Disperbyk-190, Disperbyk-191, Disperbyk-192, Disperbyk-2000, Disperbyk-2001, Disperbyk-2163, and Disperbyk-2164 (produced by BYK-Chemie), Polymer 100, Polymer 120, Polymer 150, Polymer 400, Polymer 401, Polymer 402, Polymer 403, Polymer 450, Polymer 451, Polymer 452, Polymer 453, EFKA-46, EFKA-47, EFKA-48, EFKA-49, EFKA-1501, EFKA-1502, EFKA-4540, and EFKA-4550 (produced by EFKA Chemical Corp.), Flowlen DOPA-158, Flowlen DOPA-22, Flowlen DOPA-17, Flowlen G-700, Flowlen TG-720W, Flowlen-730W, Flowlen-740W, and Flowlen 745W (produced by Kyoeisha Chemical Co., Ltd.), Ajisper PA-111, Ajisper PB-711, Ajisper PB-811, Ajisper PB-821, and Ajisper PW-911 (produced by Ajinomoto Co. Inc.), Johncryl 678, Johncryl 679, and Johncryl 62 (produced by Johnson Polymer B.V., and AQUALON KH-10, HITENOL NF-13 (produced by DAI-ICHI KOGYO SEIYAKU CO., LTD.). These products may be used alone or in combination of two or more thereof.

The case of executing surface treatment to yellow pigment microparticles will be explained hereinafter.

Surface treatment by introducing a modification group at least to a surface of yellow pigment microparticles may be done by containing a surface-modification agent into fluids to be processed which are introduced between the processing surfaces 1 and 2. The surface-modification agent may be contained in any one of the fluid which contains a yellow pigment solution (first fluid) and the fluid which contains a solvent capable of being a poor solvent to a yellow pigment (second fluid) or both fluids; or alternatively, the surface-modification agent may be contained in a third fluid which is different from any of the fluid which contains a solvent capable of being the poor solvent to a yellow pigment and the fluid which contains the yellow pigment solution. Here, combination of the first fluid and the second fluid is not particularly limited to the above example.

A kind of the modification group to be introduced as a surface-modification agent to at least the pigment surface is not particularly restricted; in the case that purpose of the surface treatment is to improve dispersibility, the modification group may be selected in accordance with, for example, a solvent for intended dispersion and kind of a dispersing agent. An example of the modification group includes those having a polar group such as an acidic group and a basic group, a salt structure of the foregoing polar groups, any one of a highly polar atom such as oxygen and sulfur and a highly polarizability structure introduced with an aromatic ring and the like or both, a hydrogen-bonding group, a hetero-ring, and an aromatic ring. An example of the acidic group includes a hydroxyl group (a hydroxy group), a sulfonic acid group (a sulfo group), a carboxylic acid group, a phosphoric acid group, and a boric acid group. An example of the basic group includes an amino group. An example of the hydrogen-bonding group includes a urethane moiety, a thiourethane moiety, a urea moiety, and a thiourea moiety.

In the case that purpose of the surface treatment is other than to improve dispersibility, for example, in the case that a surface of the yellow pigment microparticles is made water-repellent, lipophilic, or compatible with an organic solvent, the surface of the yellow pigment microparticles discharged from between the processing surfaces 1 and 2 may be made lipophilic by containing a surface-modifying agent having a lipophilic functional group in any one of the first fluid and the second fluid or both so that the lipophilic functional group may be introduced as the modification group. Further, the foregoing surface-modification agent may be contained in a third fluid which is different from any of the first fluid and the second fluid.

In the case that a surface of the yellow pigment microparticles is subjected to the treatment of attaching a resin as the surface-modifying agent, at least a part of a surface of the yellow pigment microparticles discharged from between the processing surfaces 1 and 2 may be covered with the resin by containing the resin in any one of the first fluid and the second fluid or both, whereby carrying out, for example, a hydrophilic treatment. Further, the foregoing resin may be contained in a third fluid which is different from any of the first fluid and the second fluid.

The foregoing surface treatment is not limited to the case in which surface modification of the yellow pigment microparticles is done between the processing surfaces 1 and 2 as mentioned above; but also it may be done after discharge of the yellow pigment microparticles from between the processing surfaces 1 and 2. In the latter case, after the fluid which contains the yellow pigment microparticles is discharged from between the processing surfaces 1 and 2, a material to be used for surface treatment of the yellow pigment microparticles is added into this discharged fluid; and then, the surface treatment of the yellow pigment microparticles may be done by such procedure as stirring. Alternatively, after the fluid which contains the yellow pigment microparticles is discharged, impure materials are removed by a dialysis tube or the like from the fluid which contains the yellow pigment microparticles, and then, the surface treatment may be done by adding a material for the surface treatment. Further, the surface treatment may be done after the yellow pigment microperticles are made to powders by drying the liquid component of the fluid discharged from between the processing surfaces 1 and 2, the fluid containing the yellow pigment microparticles. Specifically, after the obtained powders of the yellow pigment microparticles are dispersed in an intended solvent, a material for the surface treatment is added to the resulting dispersion solution, and then, the surface treatment may be done by such procedure as stirring.

A method for producing yellow pigment microparticles in the present invention of the application (the forced ultrathin film rotary reaction method) can freely change the Reynolds number of its minute flow path and can thus form yellow pigment microparticles which are monodisperse and excellent in re-dispersibility, having an objective particle size, particle shape and crystal form. By their self-dischargeability, there is no clogging with products even in a reaction accompanied by separation, and a large pressure is not necessary. Accordingly, the method in the present invention is superior in safety, hardly mixed in with impurities, excellent in washing performance, thus can stably produce yellow pigment microparticles. In addition, the method can be scaled up depending on the intended amount of production, thus can provide a highly productive method for producing yellow pigment microparticles.

A yellow pigment composition according to the present invention relates to a blue color, and it can be used in a wide range for, for example, a coating material, an inkjet ink, a thermal transfer ink, a toner, a colored resin, and a color filter.

### EXAMPLES

Hereinafter, the present invention will be explained by Examples of producing; the yellow pigment microparticles by using an apparatus based on the same principle as disclosed in the Patent Document 4 filed by the Applicant of the present invention, wherein, in the yellow pigment microparticles, difference between the maximum transmittance (Tmax) and the minimum transmittance (Tmin) in 350 nm to 800 nm of the transmission spectrum thereof (Tmax−Tmin) is 80% or more. However, the present invention is not limited to the following Examples.

By using the apparatus as shown in FIG. 1 wherein uniform stirring and mixing are done in a thin film fluid formed between the processing surfaces 1 and 2 which are disposed in a position they are faced with each other so as to be able to approach to and separate from each other, at least one of which rotates relative to the other, an isoindoline yellow pigment (C. I. Pigment Yellow 185; hereinafter PY-185) solution (yellow pigment solution) having a PY-185 pigment dissolved in a solvent and a solvent capable of being a poor solvent to PY-185 to separate the PY-185 microparticles are converged and uniformly mixed in the thin film fluid thereby separating the PY-185 microparticles. In addition, by using the apparatus as shown in FIG. 1 wherein uniform stirring and mixing are done in a thin film fluid formed between the processing surfaces 1 and 2 which are disposed in a position they are faced with each other so as to be able to approach to and separate from each other, at least one of which rotates relative to the other, a disazo yellow pigment (C. I. Pigment Yellow 155; hereinafter PY-155) solution (yellow pigment solution) having a PY-155 pigment dissolved in a solvent and a solvent capable of being a poor solvent to PY-155 to separate the PY-155 microparticles are converged and uniformly mixed in the thin film fluid thereby separating the PY-155 microparticles. Further, by using the apparatus as shown in FIG. 1 wherein uniform stirring and mixing are done in a thin film fluid formed between the processing surfaces 1 and 2 which are disposed in a position they are faced with each other so as to be able to approach to and separate from each other, at least one of which rotates relative to the other, a disazo yellow pigment (C. I. Pigment Yellow 180; hereinafter PY-180) solution (yellow pigment solution) having a PY-180 pigment dissolved in a solvent and a solvent capable of being a poor solvent to PY-180 to separate the PY-180 microparticles are converged and uniformly mixed in the thin film fluid thereby separating the PY-180 microparticles.

In the following examples, the term “from the center” means “through the first introduction part d1” in the processing apparatus shown in FIG. 1, the first fluid refers to the first processed fluid, and the second fluid refers to the second processed fluid introduced “through the second introduction part d2” in the processing apparatus shown in FIG. 1. Additionally, “%” indicates “% by weight” in this context.

### (Volume-average Particle Size)

Particle size distribution was measured by using a particle size distribution measuring instrument (trade name: Nanotrac UPA-UT151, produced by Nikkiso Co., Ltd.), and the volume-average particle size was adopted.

### (Powder X-ray Diffraction: XRD)

Powder X-ray Diffraction was measured by a full-automatic multipurpose X-ray diffraction instrument (trade name: X'Pert PRO MPD, produced by PANalytical B.V.). Diffraction intensity was measured within a range of diffractin angle 2 theta=5 degree to 50 degree.

### (Transmission Spectrum And Absorption Spectrum)

Transmission spectrum and absorption spectrum in a wavelength range of 350 nm to 800 nm was measured with a UV visible spectrophotometer UV-2450 (produced by Shimadzu Corp.).

### (Fluorescence Spectrum)

Three-dimensional measurement of a fluorescence spectrum was done by using a spectrophotofluorometer FP-6500 (produced by JASCO Corp.). For measurement of powders, a FDA-430 high sensitive cell holder was used; and for measurement of dispersion solutions, a 10-mm square cell was used.

### Examples 1 to 6

Pure water, an aqueous citric acid solution, or methanol was introduced as a first fluid from the center into between the processing surfaces 1 and 2 with supply pressure of 0.30 MPaG and rotation speed of 300 rpm to 3600 rpm, together with, as a second fluid, a PY-185 solution (yellow pigment solution) having PY-185 dissolved in concentrated sulfuric acid (98%) or in a mixed solvent of dimethyl sulfoxide with potassium hydroxide-containing ethanol. A dispersion solution of the PY-185 microparticles was discharged from between the processing surfaces 1 and 2. The discharged PY-185 microparticles were loosely aggregated, collected by a filter cloth and an aspirator, and then washed by pure water. Finally obtained paste of the PY-185 microparticles was dried at 30° C. under vacuum of −0.1 MPaG. XRD of powders of the PY-185 microparticles after drying was measured. The paste of the PY-185 microparticles before drying was subjected to dispersion treatment in a dispersant medium of pure water which contained Neogen R-K (active ingredient of sodium dodecylbenzenesulfonate, produced by Dai-Ichi Kogyo Seiyaku Co., Ltd.) as a surfactant. The dispersion solution of the PY-185 microparticles after the dispersion treatment was subjected to measurement of the particle diameter distribution thereof by using pure water as a solvent. Part of the aqueous dispersion solution of the PY-185 microparticles was diluted by pure water; and then, transmission spectrum of the dispersion solution thereof with the PY-185 concentration of 0.003% by weight was measured. Transmission spectra of the dispersion solutions of the PY-185 microparticles prepared in Examples 1 to 3 are shown in FIG. 4.

In Examples 1 to 6, kinds of the first fluid and the second fluid, rotation speed, temperature of the supplied solution (temperature just before introduction of the respective fluids into the processing apparatus), and introducing rate (flow amount) (unit: mL/minute) were changed as shown in Table 1. The results as to the volume-average particle diameter by particle diameter distribution measurement and difference (Tmax−Tmin) between the maximum transmittance (Tmax) and the minimum transmittance (Tmin) in 350 nm to 800 nm of the transmission spectrum in the dispersion solutions of the PY-185 microparticles prepared in Examples 1 to 6 are shown in Table 1. A TEM picture of the PY-185 microparticles prepared in Example 1 is shown in FIG. 5. It can be seen that form of the PY-185 microparticles thereby obtained is almost spherical. In FIG. 6, powder X-ray diffraction spectrum of the PY-185 microparticles prepared in Example 1 is shown in (A), powder X-ray diffraction spectrum of the PY-185 microparticle powders prepared in Example 2 is shown in (B), and powder X-ray diffraction spectrum of PY-185 used as a starting raw material in the second fluid is shown in (C). As can be seen in Table 1 and FIG. 4 to FIG. 6, what could be provided in the present invention are: a pigment composition containing at least one kind of the PY-185 microparticle, wherein difference (Tmax−Tmin) between the maximum transmittance (Tmax) and the minimum transmittance (Tmin) in 350 nm to 800 nm of the transmission spectrum thereof is 80% or more; and a method for producing the PY-185 microparticles. In other words, a pigment composition containing at least one kind of the PY-185 microparticle having spectral characteristics almost equivalent to the required spectral characteristics of the yellow color described in Patent Document 1 and Patent Document 2, and a method for producing the PY-185 microparticles could be provided. Further, the PY-185 microparticles, which constitute the PY-185 pigment composition, having the volume-average particle diameter being 1 nm to 200 nm, with the particle diameter thereof being controlled, could be prepared; and thus, expression of the color characteristics such as intended color tone and coloring power can be expected.

In FIG. 7 to FIG. 9, measurement results of the fluorescence spectra are shown, though these do not restrict the present invention. As a result of the three-dimensional measurement, in both the PY-185 microparticle powders obtained in Example 1 and the PY-185 powders used as a starting raw material in the second fluid, fluorescence light was observed in wavelength region of ca. 500 nm to ca. 700 nm with exciting wavelength region of 220 nm to 640 nm. However, when comparison was made between fluorescence spectrum of the PY-185 microparticle powders obtained in Example 1 at the exciting wavelength of 400 nm (FIG. 7) and fluorescence spectrum of the PY-185 powders used as a starting raw material in the second fluid at the exciting wavelength of 400 nm (FIG. 8), peak position of the fluorescence spectrum of the PY-185 microparticle powders obtained in Example 1 is located at 560 nm, while peak position of the fluorescence spectrum of the PY-185 powders used as a starting raw material in the second fluid is located at 535 nm, showing that the fluorescence spectrum peak of the PY-185 microparticle powders obtained in Example 1 is shifted significantly to the longer wavelength side (red shift) as compared with the fluorescence spectrum peak of the PY-185 powders used as a starting raw material in the second fluid. Shift of the peak position like this was recognized not only in the case that the exciting wavelength of 400 nm was used but also in the case that the exciting wavelength of 220 nm to 500 nm was used. In addition, when fluorescence spectrum of the dispersion solution of the PY-185 microparticles prepared in Example 1 (PY-185 concentration of 0.003% by weight) was measured, fluorescence light was observed in wavelength region of ca. 500 nm to ca. 670 nm with the exciting wavelength region of 220 nm to 700 nm. In FIG. 9, fluorescence spectrum of dispersion solution of the PY-185 microparticles prepared in Example 1 with exciting wavelength of 400 nm is shown. On the contrary, fluorescence light could not be recognized in dispersion solution of PY-185 used as a starting raw material in the second fluid (PY-185 concentration of 0.003% by weight) . Meanwhile, dispersion solution of PY-185 used as a starting raw material in the second fluid (PY-185 concentration of 0.003% by weight) was prepared similarly to the dispersion solutions of the PY-185 microparticles prepared in Examples 1 to 6 (PY-185 concentration of 0.003% by weight); namely, the PY-185 powders were subjected to dispersion treatment in a dispersant medium of pure water which contained Neogen R-K (active ingredient of sodium dodecylbenzenesulfonate, produced by Dai-Ichi Kogyo Seiyaku Co., Ltd.) as a surfactant.

TABLE 1

| | | | | | | | | Particle | |
|---|---|---|---|---|---|---|---|---|---|
| | | | | | | | | diameter | |
| | | | | | | | | measurement | |
| | | | | | | | | result | Transmission |
| | Rotation | First fluid | | | Second fluid | | | Volume-average | spectrum |
| | speed | | Flow rate | Temp | | Flow rate | Temp | particle diameter | Tmax-Tmin |
| Example | (rpm) | Solvent | (mL/min) | (° C.) | Solvent | (mL/min) | (° C.) | (nm) | (%) |
| 1 | 1700 | Pure water | 400 | 5 | 4% PY-185/(98% conc. | 3 | 25 | 11.4 | 98.2 |
| 2 | 1700 | Pure water | 200 | 5 | sulfuric acid) | 5 | 25 | 8.9 | 97.5 |
| 3 | 1700 | Pure water | 200 | 25 | | 5 | 25 | 30.3 | 96.3 |
| 4 | 3600 | Pure water | 100 | 40 | | 10 | 40 | 112.3 | 91.6 |
| 5 | 300 | Methanol | 200 | 50 | | 3 | 45 | 164.3 | 83.5 |
| 6 | 1700 | 1% Aqueous | 400 | 5 | 2% PY-185/98% (80% | 5 | 25 | 30.6 | 99.4 |
| | | citric acid | | | DMSO + 20% 0.5N—KOH—EtOH) | | | | |
| | | solution | | | | | | | |

### Examples 7 to 9

Methanol, or a mixed solvent of methanol with acetic acid was introduced as a first fluid from the center into between the processing surfaces 1 and 2 with supply pressure of 0.30 MPaG and rotation speed of 1700 rpm, together with, as a second fluid, a PY-155 solution (yellow pigment solution) having PY-155 dissolved in concentrated sulfuric acid (98%) or in a mixed solvent of dimethyl sulfoxide with potassium hydroxide-containing ethanol. A dispersion solution of the PY-155 microparticles was discharged from between the processing surfaces 1 and 2. The discharged PY-155 microparticles were loosely aggregated and then spun down by centrifugal separation (×26000 G). Supernatant after the centrifugal separation was removed; and then, after the PY-155 microparticles were dispersed by adding pure water, centrifugal separation was repeated to wash the PY-155 microparticles. Finally obtained paste of the PY-155 microparticles was dried at 30° C. under vacuum of −0.1 MPaG. XRD of powders of the PY-155 microparticles after drying was measured. The paste of the PY-155 microparticles before drying was subjected to dispersion treatment in a dispersant medium of pure water which contained sodium dodecylsulfate (SDS, produced by Kanto Chemical Co., Inc.) as a surfactant. The dispersion solution of the PY-155 microparticles after the dispersion treatment was subjected to measurement of the particle diameter distribution thereof by using pure water as a solvent. Part of the aqueous dispersion solution of the PY-155 microparticles was diluted by pure water; and then, transmission spectrum of the dispersion solution thereof with the concentration of PY-155 being 0.004% by weight was measured. Transmission spectra of the dispersion solutions of the PY-155 microparticles prepared in Example 7 and Example 8 are shown in FIG. 10. In FIG. 11, the absorption spectrum of dispersion solution of the PY-155 microparticles prepared in Example 7 (pigment concentration of 0.0014% by weight) is shown, though this does not particularly restrict the present invention.

In Examples 7 to 9, kinds of the first fluid and the second fluid, temperature of the supplied solution (temperature just before introduction of the respective fluids into the processing apparatus), and introducing rate (flow amount) (unit: mL/minute) were changed as shown in Table 2. The results as to the volume-average particle diameter by particle diameter distribution measurement and difference (Tmax−Tmin) between the maximum transmittance (Tmax) and the minimum transmittance (Tmin) in 350 nm to 800 nm of the transmission spectrum in the dispersion solutions of the PY-155 microparticles prepared in Examples 7 to 9 are shown in Table 2. As can be seen in Table 2 and FIG. 10, what could be provided in the present invention are: a pigment composition containing at least one kind of the PY-155 microparticle, wherein difference (Tmax−Tmin) between the maximum transmittance (Tmax) and the minimum transmittance (Tmin) in 350 nm to 800 nm of the transmission spectrum thereof is 80% or more; and a method for producing the PY-155 microparticles. In other words, a pigment composition containing at least one kind of the PY-155 microparticle having spectral characteristics almost equivalent to the required spectral characteristics of the yellow color described in Patent Document 1 and Patent Document 2, and a method for producing the PY-155 microparticles could be provided. Further, the PY-155 microparticles, which constitute the PY-155 pigment composition, having the volume-average particle diameter being 1 nm to 200 nm, with the particle diameter thereof being controlled, could be prepared; and thus, expression of the color characteristics such as intended color tone and coloring power can be expected.

TABLE 2

| | | | | | | | | Particle | |
|---|---|---|---|---|---|---|---|---|---|
| | | | | | | | | diameter | Trans- |
| | | | | | | | | measurement | mission |
| | | | | | | | | result | spectrum |
| | Rotation | First fluid | | | Second fluid | | | Volume-average | Tmax- |
| | speed | | Flow rate | Temp | | Flow rate | Temp | particle | Tmin |
| Example | (rpm) | Solvent | (mL/min) | (° C.) | Solvent | (mL/min) | (° C.) | diameter (nm) | (%) |
| 7 | 1700 | Methanol | 400 | 25 | 2% PY-155/(98% conc. | 3 | 25 | 18.3 | 98.7 |
| 8 | 1700 | Methanol | 200 | 25 | sulfuric acid) | 5 | 25 | 8.9 | 98.2 |
| 9 | 1700 | 1% Acetic acid/ | 200 | −10 | 2% PY-155/98% (90% | 5 | 25 | 11.3 | 99.4 |
| | | 99% methanol | | | DMSO + 10% 0.5N—KOH—EtOH) | | | | |

### Examples 10 to 12

An aqueous citric acid solution or methanol was introduced as a first fluid from the center into between the processing surfaces 1 and 2 with supply pressure of 0.30 MPaG and rotation speed of 1700 rpm, together with, as a second fluid, a PY-180 solution (yellow pigment solution) having PY-180 dissolved in concentrated sulfuric acid (98%) or in a mixed solvent of dimethyl sulfoxide with potassium hydroxide-containing ethanol. A dispersion solution of the PY-180 microparticles was discharged from between the processing surfaces 1 and 2. The discharged PY-180 microparticles were loosely aggregated, collected by a filter cloth and an aspirator, and then washed by pure water. Finally obtained paste of the PY-180 microparticles was subjected to dispersion treatment in a dispersant medium of pure water which contained Neogen R-K (active ingredient of sodium dodecylbenzenesulfonate, produced by Dai-Ichi Kogyo Seiyaku Co., Ltd.) as a surfactant. The dispersion solution of the PY-180 microparticles after the dispersion treatment was subjected to measurement of the particle diameter distribution thereof by using pure water as a solvent. Part of the aqueous dispersion solution of the PY-180 microparticles was diluted by pure water; and then, transmission spectrum of the dispersion solution thereof with the concentration of the PY-180 being 0.004% by weight was measured. Transmission spectra of the dispersion solutions of PY-180 microparticles prepared in Examples 10 and 11 are shown in FIG. 12.

In Examples 10 to 12, kinds of the first fluid and the second fluid, temperature of the supplied solution (temperature just before introduction of the respective fluids into the processing apparatus), and introducing rate (flow amount) (unit: mL/minute) were changed as shown in Table 3. The results as to the volume-average particle diameter by particle diameter distribution measurement and difference (Tmax−Tmin) between the maximum transmittance (Tmax) and the minimum transmittance (Tmin) in 350 nm to 800 nm of the transmission spectrum in the dispersion solutions of the PY-180 microparticles prepared in Examples 10 to 12 are shown in Table 3. As can be seen in Table 3 and FIG. 12, what could be provided in the present invention are: a pigment composition containing at least one kind of the PY-180 microparticle, wherein difference (Tmax−Tmin) between the maximum transmittance (Tmax) and the minimum transmittance (Tmin) in 350 nm to 800 nm of the transmission spectrum thereof is 80% or more; and a method for producing the PY-180 microparticles. In other words, a pigment composition containing at least one kind of the PY-180 microparticle having spectral characteristics almost equivalent to the required spectral characteristics of the yellow color described in Patent Document 1 and Patent Document 2, and a method for producing the PY-180 microparticles could be provided. Further, the PY-180 microparticles, which constitute the PY-180 pigment composition, having the volume-average particle diameter being 1 nm to 200 nm, with the particle diameter thereof being controlled, could be prepared; and thus, expression of the color characteristics such as intended color tone and coloring power can be expected.

TABLE 3

| | | | | | | | | Particle | |
|---|---|---|---|---|---|---|---|---|---|
| | | | | | | | | diameter | |
| | | | | | | | | measurement | |
| | | | | | | | | result | Transmission |
| | Rotation | First fluid | | | Second fluid | | | Volume-average | spectrum |
| | speed | | Flow rate | Temp | | Flow rate | Temp | particle diameter | Tmax-Tmin |
| Example | (rpm) | Solvent | (mL/min) | (° C.) | Solvent | (mL/min) | (° C.) | (nm) | (%) |
| 10 | 1700 | 1% Aqueous citric | 200 | 5 | 2% PY-180/98% (80% | 5 | 25 | 26.4 | 99.1 |
| | | acid solution | | | DMSO + 20% | | | | |
| 11 | 1700 | Methanol | 400 | 25 | 0.5N—KOH—EtOH) | 3 | 25 | 6.8 | 96.7 |
| 12 | 1700 | Methanol | 200 | 25 | 2% PY-180/(98% conc. | 5 | 25 | 20.3 | 98.4 |
| | | | | | sulfuric acid) | | | | |

### EXPLANATION OF REFERENCE NUMERALS

1 first processing surface

2 second processing surface

10 first processing member

11 first holder

20 second processing member

21 second holder

23 separation-regulating surface

d1 first introduction part

d2 second introduction part

d20 opening

p fluid pressure imparting mechanism

1. A yellow pigment composition containing at least one kind of yellow pigment microparticle, wherein difference (Tmax−Tmin) between the maximum transmittance (Tmax) and the minimum transmittance (Tmin) of a transmission spectrum thereof in a region of 350 nm to 800 nm is 80% or more.

2. The yellow pigment composition according to claim 1, wherein the yellow pigment microparticle is an organic pigment.

3. The yellow pigment composition according to claim 1, wherein the yellow pigment microparticle is an azo pigment or an isoindoline pigment.

4. The yellow pigment composition according to claim 1, wherein the yellow pigment microparticle is formed by a process comprising:
a fluid to be processed is supplied between processing surfaces being capable of approaching to and separating from each other and displacing relative to each other,
pressure of force to move in the direction of approaching, including supply pressure of the fluid to be processed and pressure applied between the rotating processing surfaces, is balanced with pressure of force to move in the direction of separation thereby keeping a minute space in a distance between the processing surfaces,
the minute space kept between two processing surfaces is used as a flow path of the fluid to be processed, thereby forming a thin film fluid of the fluid to be processed, and
the microparticle is formed in this thin film fluid.

5. The yellow pigment composition according to claim 1, wherein form of the yellow pigment microparticle is almost spherical.

6. The yellow pigment composition according to claim 5, wherein a volume-average particle diameter of the yellow pigment microparticle is in the range of 1 nm to 200 nm.

7. A method to produce yellow pigment microparticles, a method to produce the yellow pigment microparticles according to claim 1, wherein:
a fluid to be processed is supplied between processing surfaces being capable of approaching to and separating from each other and displacing relative to each other,
pressure of force to move in the direction of approaching, including supply pressure of the fluid to be processed and pressure applied between the rotating processing surfaces, is balanced with pressure of force to move in the direction of separation thereby keeping a minute space in the distance between the processing surfaces,
the minute space kept between two processing surfaces is used as a flow path of the fluid to be processed, thereby forming a thin film fluid of the fluid to be processed, and
the yellow pigment microparticles are separated in this thin film fluid.

8. The method for producing yellow pigment microparticles according to claim 7, wherein the method comprises:
a fluid pressure imparting mechanism for imparting pressure to a fluid to be processed,
at least two processing members of a first processing member and a second processing member, the second processing member being capable of relatively approaching to and separating from the first processing member, and
a rotation drive mechanism for rotating the first processing member and the second processing member relative to each other; wherein
each of the processing members is provided with at least two processing surfaces of a first processing surface and a second processing surface disposed in a position they are faced with each other,
each of the processing surfaces constitutes part of a forced flow path through which the fluid to be processed under the pressure is passed,
of the first and second processing members, at least the second processing member is provided with a pressure-receiving surface, and at least part of the pressure-receiving surface is comprised of the second processing surface,
the pressure-receiving surface receives pressure applied to the fluid to be processed by the fluid pressure imparting mechanism thereby generating force to move in the direction of separating the second processing surface from the first processing surface,
the fluid to be processed under the pressure is passed between the first and second processing surfaces being capable of approaching to and separating from each other and rotating relative to each other, whereby the fluid to be processed forms the thin film fluid, and
the yellow pigment microparticles are separated in this thin film fluid.

9. The method for producing yellow pigment microparticles according to claim 8, wherein:
one kind of fluid to be processed is introduced to between the first processing surface and the second processing surface,
an another independent introduction path for another kind of fluid to be processed other than the one kind of the fluid to be processed is provided,
at least one opening leading to this introduction path is arranged in at least either one of the first processing surface or the second processing surface,
the another kind of the fluid to be processed is introduced between both the processing surfaces through this introduction path, and
the one kind of the fluid to be processed and the another kind of the fluid to be processed are mixed in the thin film fluid.

10. The method for producing yellow pigment microparticles according to claim 9, wherein:
the opening is arranged in a downstream side of a point at which the one kind of the fluid to be processed becomes a laminar flow between both the processing surfaces, and
mixing of the fluids to be processed is done by introducing the another kind of the fluid to be processed from the opening.
